(19) United States
(12) Patent Application Publication
(10) Pub. No.: US 2011/0031144 A1
(43) Pub. Date: Feb. 10, 2011

(54) WALL MOUNTABLE RETAINER FOR MEDICAL SHARPS

(76) Inventor: John Allegretti, Barrington Hills, IL (US)

Correspondence Address: GREER, BURNS & CRAIN, 300 S WACKER DR, 25TH FLOOR, CHICAGO, IL, 60606, US

(21) Appl. No.: 12/536,860

(22) Filed: Aug. 6, 2009

(51) Int. Cl. A61B 19/02; B65D 25/00; B65D 25/28

(52) U.S. Cl. 206/366; 220/694; 220/752

(57) ABSTRACT

A retainer having a waste container for disposing medical sharps, the retainer including a housing defining a chamber, the waste container being disposed in the chamber, an opening disposed on the housing and above the chamber, a receiving device having an open upper end and a lower end which engages the opening, and a guide plate having a solid portion and an aperture and being disposed in the chamber, the guide plate being constructed and arranged to move by user actuation between a closed position wherein at least a portion of the solid portion is generally aligned with the opening and an open position wherein the aperture is generally aligned with the opening.

### BACKGROUND

The present invention relates to a retainer for medical waste, and more particularly to a wall mountable retainer for receiving disposed medical sharps.

Medical needles, syringes, sharps, and the like (collectively referred to as “sharps”) are commonly used to administer medications as well as manage medical conditions such as diabetes, allergies, arthritis, asthma, infertility, hepatitis, and HIV/AIDS. Disposal of used sharps is an ongoing issue at medical facilities. In addition to the use by medical personnel, individuals with such ailments often need to self-inject or test while at work or in other public places. Most of the sharps used for these latter injections end up discarded in community solid waste receptacles, putting workers and the public at risk of sharp stick injuries and potentially fatal infections. Sharps from illicit drug use are also dropped in the trash, thrown in toilets or left in public restrooms. Used sharps are generally considered a biohazard, and therefore it is preferred that users have an effective way to properly dispose of them.

### SUMMARY

The above-listed needs are met or exceeded by the present retainer for disposed medical sharps, which features a preferably tubular transparent sharps receiving device having an open upper end and a lower end in communication with an opening of a box-like housing for a removable medical waste container. A used sharp is dropped into the receiving device. A guide plate in the container has both a solid portion and an aperture, and is movable by a user in a one-handed operation between a closed position blocking movement of sharps in the receiving device, and an open position wherein the aperture is generally aligned with the opening to permit the sharps to fall into the waste container.

More specifically, the present retainer for disposing medical sharps includes a housing defining a chamber, a waste container disposed in the chamber, an opening disposed on the housing and above the chamber, a receiving device having an open upper end and a lower end which engages the opening, and a guide plate having a solid portion and an aperture and being disposed in the chamber. The guide plate is constructed and arranged to move by user actuation between a closed position wherein at least a portion of the solid portion is generally aligned with the opening and an open position wherein the aperture is generally aligned with the opening.

In another embodiment, a retainer for disposing medical sharps includes a housing defining a chamber, a waste container disposed in the chamber, an opening disposed on the housing and above the chamber, a receiving device having an open upper end and lower end which engages the opening, the receiving device being configured for temporarily holding at least one of the medical sharps, and a guide plate having a solid portion and an aperture and being disposed in the chamber. The guide plate is constructed and arranged to move by user actuation between a closed position, wherein at least a portion of the solid portion is generally aligned with the opening, and an open position wherein the aperture is generally aligned with the opening. The one medical sharp moves from the receiving device to the waste container upon movement of the guide plate from the closed position to the open position.

### BRIEF DESCRIPTION OF THE DRAWINGS

FIG. 1 is a front perspective view of the present retainer device with the guide plate in the closed position and the door in the closed position;

FIG. 2 is a front perspective view of the present retainer device with the guide plate in the closed position and the door in the open position;

FIG. 3 is a fragmentary front view of the present retainer device with the guide plate in the closed position and the door in the open position;

FIG. 4 is a fragmentary front view of the present retainer device with the guide plate in the open position and the door in the open position;

FIG. 5 is a bottom view of the retainer of FIG. 3, with portions removed for clarity;

FIG. 6 is a bottom view of the retainer of FIG. 4, with portions removed for clarity;

FIG. 7 is a fragmentary top view of the retainer of FIG. 3; and

FIG. 8 is a fragmentary top view of the retainer of FIG. 4.

### DETAILED DESCRIPTION

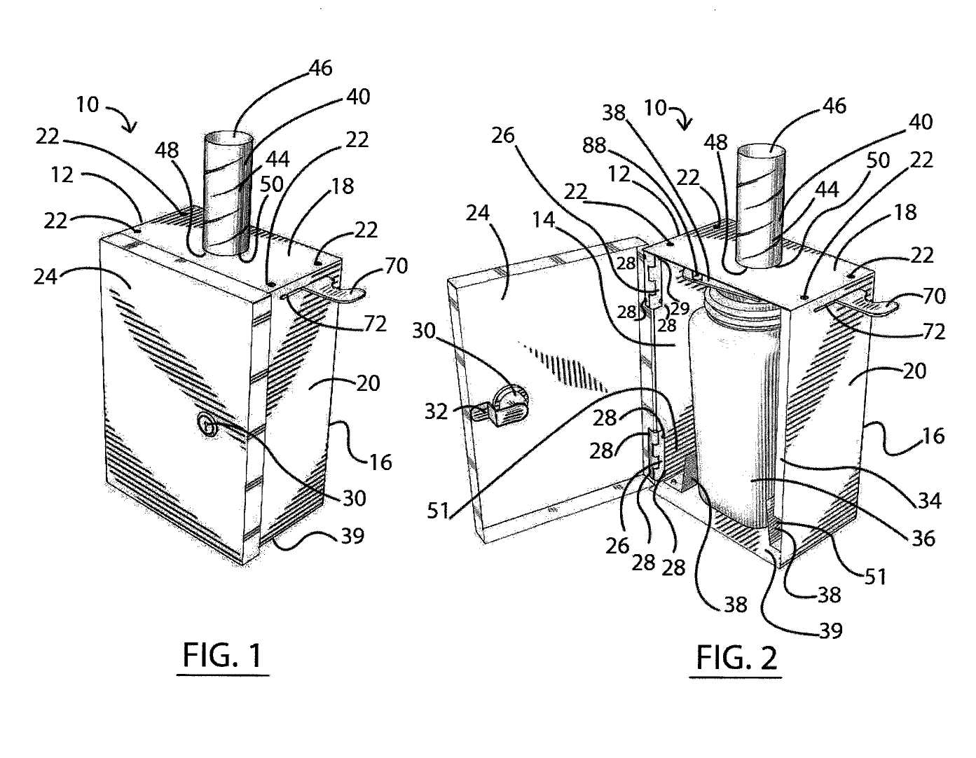

Throughout this application, use of “a,” “the,” or “said” refers to at least one unless otherwise stated. Referring now to FIGS. 1 and 2, a retainer 10 for disposing medical sharps is shown. The retainer 10 includes a housing 12 defining a chamber 14 and having a rear wall 16 and an upper wall 18 affixed to two side panels 20 using fasteners 22. At least one, but preferably two holes (not shown) are included on the rear wall 16 of the housing 12 for receiving fasteners to secure the retainer 10 to a wall. It is noted that other wall mounting techniques as known in the art are also contemplated for securing the retainer 10 to a wall. Further, unless otherwise stated, all other fasteners described in this application are preferably rivets, although other types such as screws are contemplated.

A door 24 is attached to the housing 12 using hinges 26 and fasteners 28 and is constructed and arranged to move between a closed position (FIG. 1) and an open position (FIG. 2). Preferably, the door 24 includes a locking mechanism 30 for selectively locking the door 24 in the closed position. Upon inserting and turning a key inside the locking mechanism 30, a latch 32 engages a lip 34 disposed on at least one of the side panels 20 for securing the door 24 in the closed position. Notably, other types of locking mechanisms known in the art for locking the door 24 in the closed position are contemplated.

Opening the door 24 provides a user with access to a removable waste container 36 disposed in the chamber 14. Preferably the waste container is a standard sharps-receiving one quart container as used in the sharps disposal industry, however other shapes and sizes are contemplated. Preferably, positioning fixtures 38, such as “L”-shaped pieces of metal, are disposed on a floor 39 of the housing 12 for securing and aligning the waste container 36 within the chamber 14.

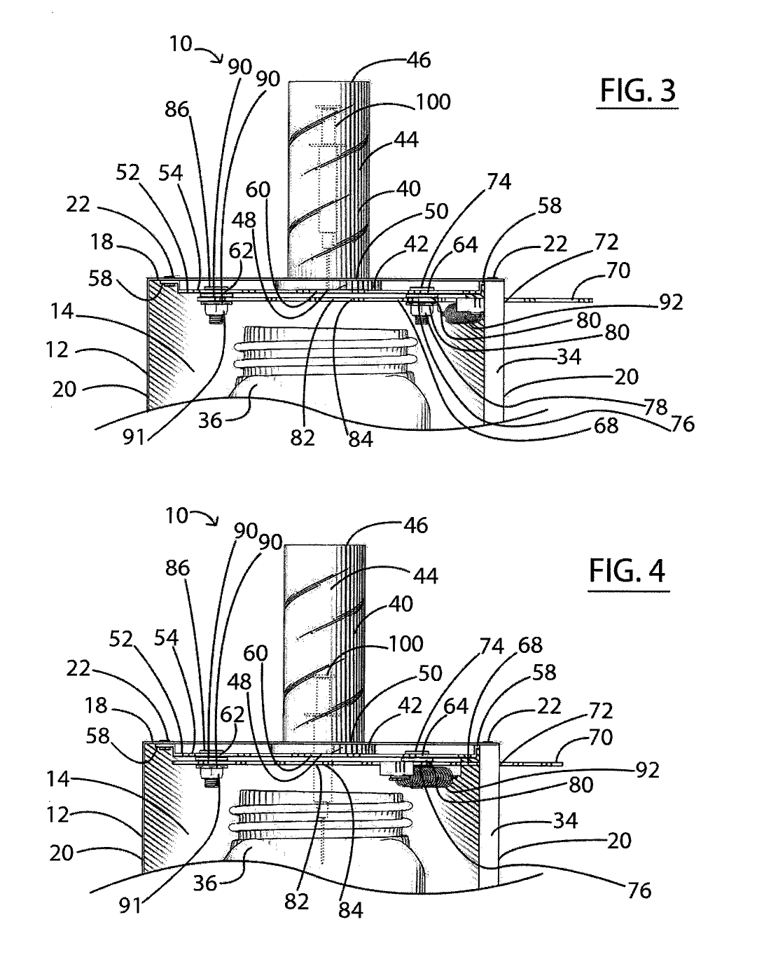

Referring now to FIGS. 3 and 4, the retainer 10 includes a receiving device 40 above the waste container 36, preferably being generally tubular or cylindrically-shaped and made of a transparent material, preferably plastic such as polycarbonate or the like. Included on the receiving device 40 is a base 42 and a body 44, the base having a larger diameter than the body. The receiving device 40 has an open upper end 46 configured for receiving medial sharps or the like deposited by a user, and a lower end 48 engaging and extending through an opening 50 on the housing 12 above the chamber 14, preferably on the upper wall 18. Notably, the term “engages” and any variations thereof used in this application refer to an arrangement capable of providing fluid communication between the two neighboring components.

While the receiving device 40 is preferably disposed directly above the waste container 36, the invention is not limited to such an arrangement. Indeed, the receiving device 40 need only be positioned such higher than the waste container 36 such that gravity exerts a force on an object in the receiving device towards the waste container so that object falls through the opening 50 into the waste container. When the user places the waste container 36 into the chamber 14, the positioning fixtures 38 guide the waste container such that it is properly aligned under the opening 50. The positioning fixtures 38 also create compartment 51 (FIG. 2) between the positioning fixture and each side panel 20, preferably to be used for storage of a lid (not shown) of the waste container 36.

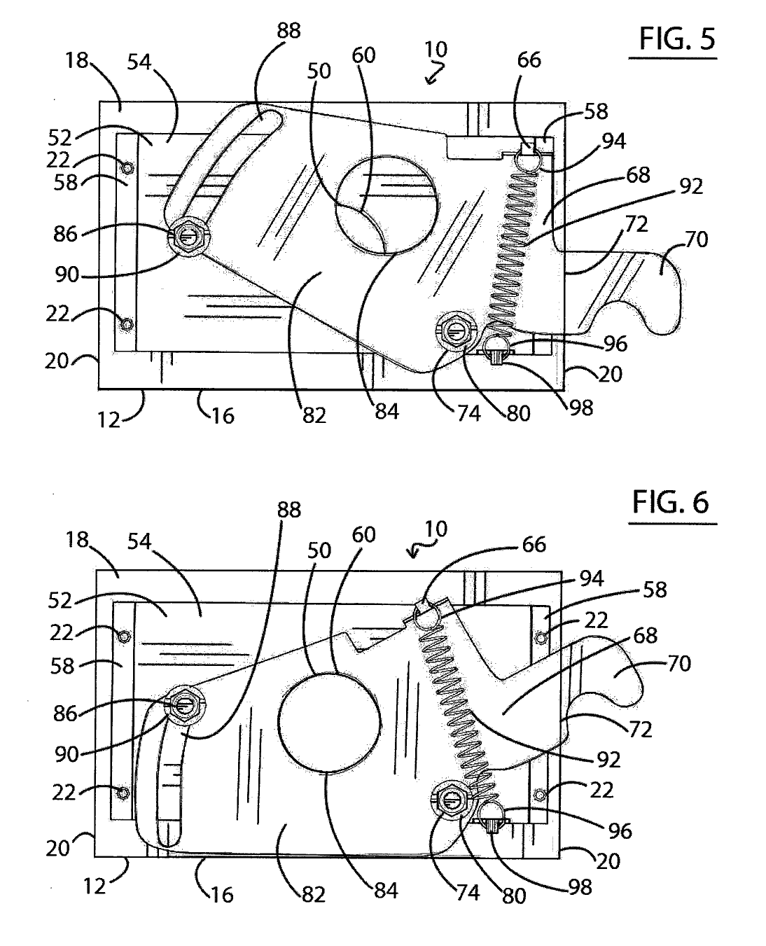

A retaining bracket 52 having a floor 54 and at least one, but preferably two flanges 58, supports the base 42 of the receiving device 40 against the housing 12 to retain the receiving device in place. The fasteners 22 attach the flanges 58 of the retaining bracket 52 to an underside of the upper wall 18 of the housing. Further included on the retaining bracket 52 is a hole 60 generally aligned with the opening 50 on the housing 12 and which is engaged by the lower end 48 of the receiving device 40. The diameter of the hole 60 is smaller than the diameter of the base 42 to prevent the base from falling through the retaining bracket 52. Further included on the retaining bracket 52 is a stopper opening 62, a pivot pin opening 64, and a hook 66 (FIGS. 5 and 6) for use in association with a guide plate 68 as described below.

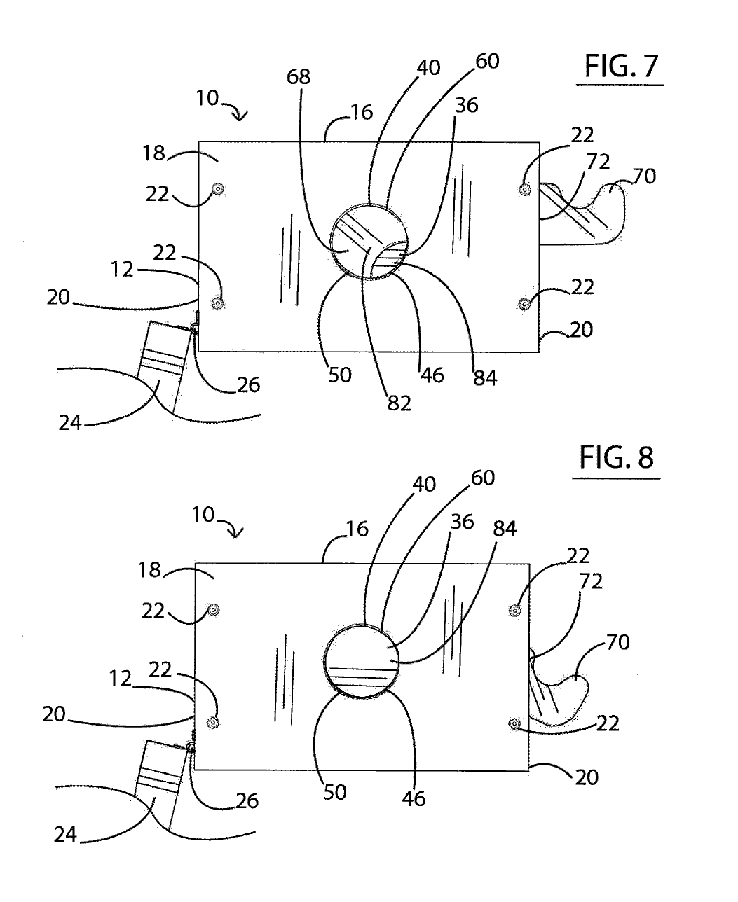

Referring now to FIGS. 3-8, the guide plate 68 is disposed in the chamber 14 between the receiving device 40 and the waste container 36, and preferably below the retaining bracket 52 such that the retaining bracket is disposed between the guide plate 68 and the upper wall 18 of the housing 12. A handle 70 is included on the guide plate 68 and extends outside of the housing 12 though a slot 72 (FIGS. 1 and 2). A pivot pin, such as a bolt, secures the guide plate to 68 to the retainer bracket 52 by extending through the pivot pin opening 64 of the retainer bracket 52 and through a pivot pin aperture 76 in the guide plate 68. A nut 78 and at least one, but preferably two washers 80 secure the pivot pin 74 in place and pivotally secure the guide plate 68 to the retainer bracket 52.

The guide plate 68 further includes a solid portion 82 and an aperture 84, and is constructed and arranged to move relative to the housing 12 about the pivoting member 74 by user actuation of the handle 70 between a closed position (FIGS. 3, 5, and 7) wherein at least a portion of the solid portion 82 is generally aligned with the opening 50, and an open position (FIGS. 4, 6, and 8), wherein the aperture 84 is generally aligned with the opening 50.

Movement of the guide plate 68 about the pivoting member 74 is limited by a stopping device 86, such as a bolt, connected to the retaining bracket 52, and which extends through a generally arcuate-shaped window 88 on the guide plate. A friction reducing member on at least one of the stopping device 86 or the window 88 facilitate the pivoting movement. In the preferred embodiment, at least one but preferably two washers 90 act as friction reducing members. Further, a nut 91 secures the stopping device 86 in place.

A biasing device 92 is connected to the housing 12 and to the guide plate 68 for urging the guide plate towards the closed position. Notably, use of the terms “attached,” “connected,” “associated,” or variations thereof throughout this application should be interpreted to include not only direct, but also indirect relationships among components. Preferably, the biasing device 92 is a spring having a first end 94 engaging the hook 66 on the housing 12 and a second end 96 engaging a tab 98 on the guide plate 68. Other connection points for the spring 92 are contemplated.

As shown in FIG. 3, when a user initially deposits a medial sharp 100 into the upper end 46 of the receiving device 40, the sharp is temporarily maintained in the receiving device since the guide plate 38 is by default in the closed position with the solid portion 82 obstructing and preventing preventing the sharp from falling though the opening 50 in the housing 12 or the hole 60 in the retaining bracket 52. However, as shown in FIG. 4, when the user actuates the handle 70, moving the guide plate 68 from the closed position to the open position, the solid portion 82 obstruction is removed and the aperture 84 becomes aligned with the opening 50 and the hole 60. Therefore, the medical sharp 100 falls from the receiving device 40, through the opening 50, the hole 60, and the aperture 84, and into the waste container 36. The preferably transparent material of the receiving device 40 allows the user to confirm that the sharp 100 has been successfully disposed.

As such, the retainer 10 can be operated by a single hand. Indeed, the user can place the sharp 100 into the receiving device 40 and then, using the same hand, pull the handle 70 towards the user causing the sharp 100 to fall into the waste container 36. The user need not support the retainer to counter the force resulting from the handle 70 being pulled because the retainer 10 is mounted to the wall. As such, the wall mount counters the force and secures the retainer 10 in place.

While alternate shapes, sizes, and arrangements of the components shown in the figures are contemplated, the preferred retainer 10 is configured such that a first distance between the upper wall 18 of the housing 12 and the guide plate 68, and a second distance between the upper wall of the housing and the floor 39 of the housing have a respective measurement ratio of 1 to at least 22. This provides for a relatively small sized retainer 10 including the described features, while still being able to completely house the waste container 36.

It is expected to be seen that medical personnel and other individuals who use medical sharps will prefer use of the present retainer 10 to the other devices for disposing sharps as known in the prior art. One advantage as discussed above is that the size of the retainer is relatively small compared to other devices, while providing a greater ratio of volume in the waste container to the volume of entire the device. Further, operation of the retainer is simple and can be performed using a single hand. The ability to wall mount the device ensures that the user need not provide a countering force when actuating the handle 70. Finally, while the design allows medical sharps to be easily disposed, the use of the locking mechanism 30 prevents unauthorized individuals from easily accessing the waste container 36.

While a particular embodiment of the present wall mountable retainer device for medical sharps has been described herein, it will be appreciated by those skilled in the art that changes and modifications may be made thereto without departing from the invention in its broader aspects and as set forth in the following claims.

1. A retainer having a waste container for disposed medical sharps, the retainer comprising:
a housing defining a chamber, the waste container being disposed in said chamber;
an opening disposed on said housing and above said chamber;
a receiving device having an open upper end and a lower end which engages said opening; and
a guide plate having a solid portion and an aperture and being disposed in said chamber, said guide plate being constructed and arranged to move by user actuation between a closed position wherein at least a portion of said solid portion is generally aligned with said opening and an open position wherein said aperture is generally aligned with said opening.

2. The retainer of claim 1 wherein said guide plate is disposed between said receiving device and the waste container.

3. The retainer of claim 1 further comprising a biasing device connected to said housing and to said guide plate for urging said guide plate towards said closed position.

4. The retainer of claim 3 wherein said biasing device is a spring having a first end engaging a hook on said housing and a second end engaging a tab on said guide plate.

5. The retainer of claim 1 wherein said guide plate pivots relative to said housing about a pivot pin and includes a handle configured for pivotally moving said guide plate between said closed position and said open position.

6. The retainer of claim 5 wherein said housing includes a slot and said handle extends through said slot and outside said housing.

7. The retainer of claim 5 further including a stopping device connected to said housing, and which extends through a generally arcuate-shaped window on said guide plate.

8. The retainer of claim 7 further including a friction reducing member on at least one of said stopping device or said window for facilitating said pivoting movement.

9. The retainer of claim 1 wherein said receiving device is generally tubular and cylindrically-shaped.

10. The retainer of claim 1 wherein said receiving device is made of a transparent material.

11. The retainer of claim 1 wherein said housing includes a door constructed and arranged to move between an open position and a closed position.

12. The retainer of claim 1 further comprising at least one positioning fixture configured for securing and aligning the waste container within said chamber and below said receiving device.

13. The retainer of claim 1 wherein said receiving device has a body and a base having a larger diameter than said body, wherein said retainer further includes a retaining bracket supporting said base against said housing to retain said receiving device in place.

14. The retainer of claim 13 wherein said retaining bracket is disposed between said guide plate and an upper wall of said housing.

15. The retainer of claim 1 wherein a first distance between an upper wall of said housing and said guide plate, and a second distance between said upper wall of said housing and a floor of said housing have a respective measurement ratio of 1 to at least 22.

16. A retainer having a waste container for disposed medical sharps, the retainer comprising:
a housing defining a chamber, the waste container being disposed in said chamber;
an opening disposed on said housing and above said chamber;
a receiving device having an open upper end and lower end which engages said opening, said receiving device being configured for temporarily holding at least one of the medical sharps; and
a guide plate having a solid portion and an aperture and being disposed in said chamber, said guide plate being constructed and arranged to move by user actuation between a closed position wherein at least a portion of said solid portion is generally aligned with said opening and an open position wherein said aperture is generally aligned with said opening, wherein the at least one medical sharp moves from the receiving device to the waste container upon movement of said guide plate from said closed position to said open position.

17. The retainer of claim 16 wherein said receiving device is disposed substantially outside of said housing.

18. The retainer of claim 16 wherein said receiving device is made of a transparent material.

19. The retainer of claim 16 further comprising a spring for urging said guide plate towards said closed position.

20. The retainer of claim 16 wherein said guide plate pivots relative to said housing about a pivot point and includes a handle configured for pivotally moving said guide plate between said closed position and said open position.
